(19) United States
(12) Patent Application Publication
(10) Pub. No.: US 2013/0226292 A1
(43) Pub. Date: Aug. 29, 2013

(54) ISLET STORING INTRAOCULAR IMPLANT, METHOD OF USING THE SAME AND KIT INCLUDING THE SAME

(71) Applicants: Jean-Marie Parel, Miami Shores, FL (US); Sonia Yoo, Pinecrest, FL (US); Andres Bernal, Sunny Isles, FL (US); William Lee, North Miami, FL (US)

(72) Inventors: Jean-Marie Parel, Miami Shores, FL (US); Sonia Yoo, Pinecrest, FL (US); Andres Bernal, Sunny Isles, FL (US); William Lee, North Miami, FL (US)

(21) Appl. No.: 13/878,978

(22) PCT Filed: Oct. 10, 2011
(86) PCT No.: PCT/US11/55632

§ 371 Date: May 16, 2013

Related U.S. Application Data
(60) Provisional application No. 61/392,234, filed on Oct. 12, 2010.

(51) Int. Cl. A61F 2/16

(52) U.S. Cl. CPC A61F 2/16; A61F 2/1662
USPC 623/6.12; 623/6.54

(57) ABSTRACT

An intraocular implant that has an anterior portion, at least one haptic element designed to localize the intraocular implant within an eye, a posterior portion, and an islet storage feature coupled to the anterior and posterior portions is disclosed. The interior of the islet storage feature is accessible from the anterior portion. The intraocular implant can be used as part of a method for treating a disease state, including, but not limited to, diabetes, diabetic retinopathy, macular degeneration, macular edema, and other blinding eye diseases.

### BACKGROUND OF THE INVENTION

1. Technical Field

The present invention relates to systems and methods for implanting islet cells.

2. Related Art

There is a need for systems and methods for delivering insulin to diabetic patients. In recent years research has been done on various systems and methods for implanting islet cells into the patient. Such systems and methods hold promise to reduce or even eliminate the frequency and amount of insulin injections that are necessary.

### SUMMARY OF THE INVENTION

An intraocular implant has an anterior portion, at least one haptic element designed to localize the intraocular implant within an eye, a posterior portion, and an islet storage feature coupled to the anterior portion and the posterior portion. An interior of the islet storage feature can be accessible from the anterior portion.

The at least one haptic element can include at least two resilient arms extending from a core of the anterior portion. The anterior portion can include an inner rim coupled to an outer rim encircling the inner rim, wherein an interior of the islet storage feature is accessible through at least one space between the inner rim and the outer rim. The inner rim and the outer rim can be circular and arranged concentrically.

An inner portion of said islet storage feature can be coupled to the inner rim and an outer portion of the islet storage feature can be coupled to the outer rim. The islet storage feature can be closed at the posterior portion. The islet storage feature can comprise a permeable material. In one aspect, the islet storage feature does not extend into an optical cavity of the intraocular implant. The intraocular implant can further comprise an optical element.

An intraocular implant kit includes an intraocular implant having an anterior portion, at least one haptic element designed to localize the intraocular implant within an eye, a posterior portion, and an islet storage feature coupled to the anterior portion and the posterior portion. The intraocular implant can be disposed within a cannula. The intraocular implant kit can further include an optical element. The optical element can be an intraocular lens. The intraocular implant kit can include a plurality of islets.

A method of treating a disease state includes the step of implanting an intraocular implant into an eye of a patient. The intraocular implant comprises an anterior portion, a posterior portion and an islet storage feature coupled to the anterior portion and the posterior portion. A plurality of islets can be deposited into an interior of the islet storage feature.

The intraocular implant can be implanted using a cannula. An optical element can be coupled to the anterior portion. An iris of the eye can be cut to form at least two iris segments. A portion of the at least two iris segments can be located in the interior of the islet storage feature. The cutting step can include cutting the iris without cutting to a root of the iris. The anterior portion can include an inner rim coupled to an outer rim encircling the inner rim element, and the interior is accessible through at least one space between the inner rim and the outer rim and the at least two iris segments extend through the at least one space. The disease state can be selected from the group consisting of diabetes, diabetic retinopathy.

### BRIEF DESCRIPTION OF THE DRAWINGS

A fuller understanding of the present invention and the features and benefits thereof will be obtained upon review of the following detailed description together with the accompanying drawings, in which:

FIG. 1 is a side, perspective view of an islet storing intraocular implant according to the current invention.

FIG. 2 is a front view of an islet storing intraocular implant according to the current invention.

FIG. 3 is a front, perspective view of an islet storing intraocular implant according to the current invention.

FIG. 4 is a front view of an eye with an implanted islet storing intraocular implant according to the current invention showing iris sections extending through the space between the inner and outer rims and into the interior of the islet storage element.

FIG. 5 is a cross-sectional view of the eye with the implanted of FIG. 4 taken along cut line 5-5.

FIG. 6. Pancreatic islets engrafted on the iris in the anterior chamber of the eye of a baboon. a, Digital images of the anterior chamber of the eye of a baboon showing islet grafts on the iris (right). The anterior chamber and the cornea were as clear as those of the control, non-transplanted eye (left). In the transplanted eye, the field of view was not obstructed by islets. b, Fluorescein angiography performed at post-operative day (POD) 24 revealed islet vascularization. c, Confocal image of a section of the anterior segment of the eye showing engraftment of islets on the iris. Insulin-labeled beta cells (red) and glucagon-labeled alpha cells (cyan) formed a distinct layer fully fused with the iris tissue. d, The cytoarchitecture of intraocular islet grafts was preserved as seen by a typical ratio of beta cells to alpha cells [quantified on right panel as area of cell specific staining/(area of insulin staining +area of glucagon staining)×100, (n=47 sections)]. Scale bars=50 μm (d) and 200 μm (a, b, c).

FIG. 7. Pancreatic islet transplantation into the anterior chamber of the eye improved glycemic control in a diabetic baboon. a, Daily insulin requirements before and after islet transplantation. The baboon gained weight after transplantation (blue symbols) until it was pancreatectomized (red arrow). Weight gain was seen again after a second islet transplantation (black arrow). b, Fluctuations in fasting blood glucose decreased after transplantation, increased after pancreatectomy, and stabilized again after a second islet transplantation. c, Plasma C-peptide levels increased after islet transplantation. C-peptide levels were much higher in the aqueous humor of the eye (red symbols), but co-varied with plasma c-peptide levels. d, Glycated hemoglobin (A1C) levels decreased after islet transplantation and, after the second islet transplantation, almost reached levels measured in the same baboon before diabetes was induced (dotted line). e, f, An injection of glucagon to stimulate insulin secretion (glucagon challenge) shows that the insulin secretory response was abolished after removing the intraocular islet grafts. Plasma C-peptide levels increased during a glucagon challenge before but not after removal of the transplanted eye (e). The baboon became hyperglycemic after removing the transplanted eye (f). Glycemia did not change with the glucagon challenge after removal of the eye, indicating that the baboon had lost responsiveness to glucagon.

### DETAILED DESCRIPTION OF THE INVENTION

The following explanations are intended as an overview of different aspects relevant to the invention. They are by no means intended to be limited by the examples that are given and should be interpreted to demonstrate exemplary range of possibilities and embodiments of this invention.

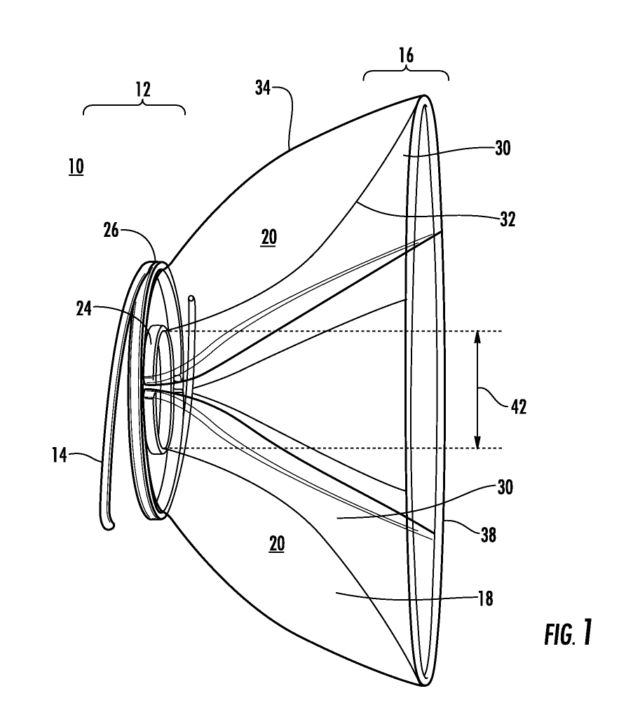

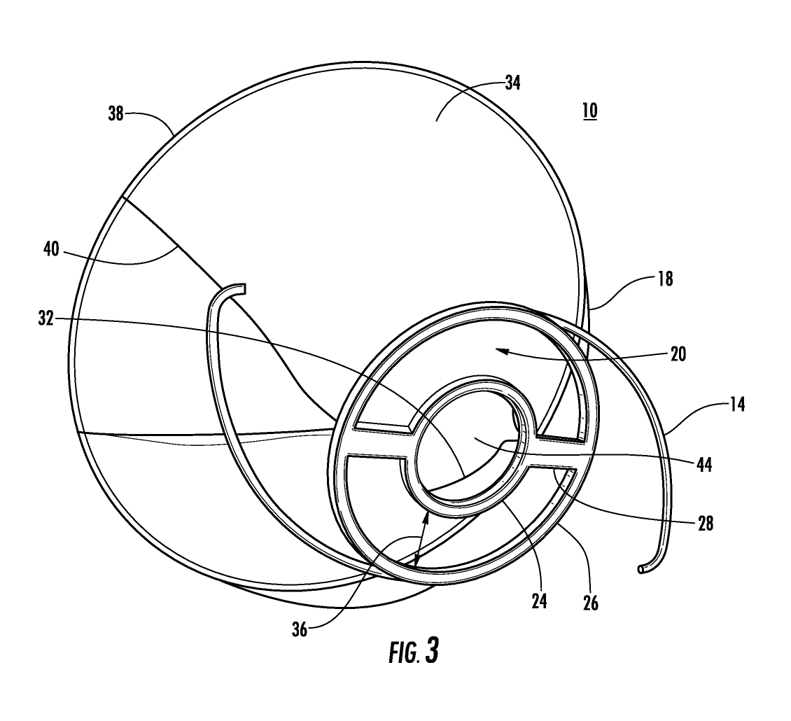

As shown in FIGS. 1-5, the invention is drawn to an intraocular implant 10 that includes an anterior portion 12, at least one haptic element 14, a posterior portion 16, and an islet storage feature 18 coupled to the anterior portion 12 and the posterior portion 16. An interior 20 of the islet storage feature 18 can be accessible from the anterior portion 12 or a posterior portion 16. As will be understood, “accessible from” has its normal meaning and includes accessible through as well as accessible around the structure of the anterior portion 12 or the posterior portion 16. The intraocular implant 10 is designed to facilitate islet transplantation procedures by including, among other features, an islet storage feature 18 designed to store pancreatic islets. As used herein, the term “islets” is used to refer to pancreatic islets, i.e., islets of Langerhans.

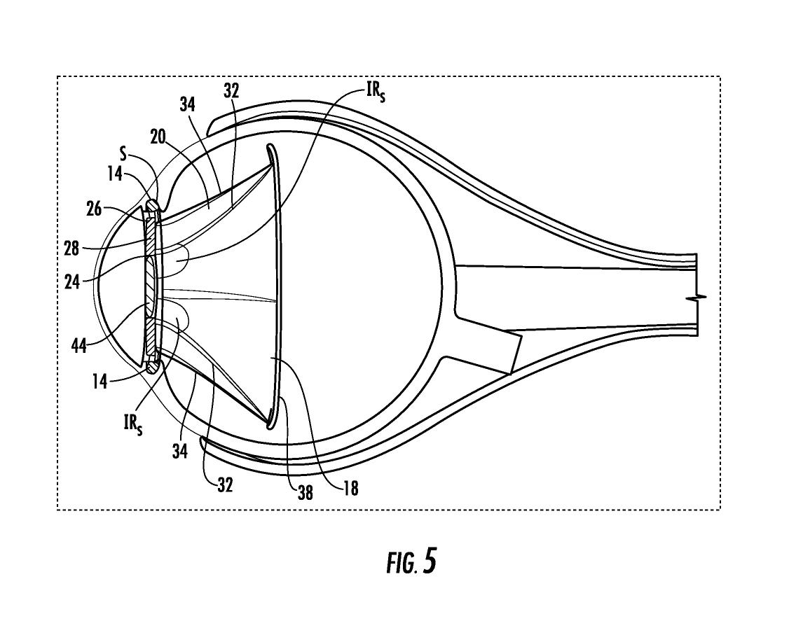

The haptic element 14 is designed to localize the intraocular implant 10 within an eye of the patient. As shown in FIG. 1, the haptic element 14 can include a pair of resilient arms extending from a core 22 of the anterior portion 12. As shown in FIG. 5, the haptic element 14 can be designed to lodge in the sulcus (S) of the eye of the patient.

The anterior portion 12 can include an inner rim 24 coupled to an outer rim 26, where the outer rim 26 encircles the inner rim 24. The inner rim 24 and the outer rim 26 can be operatively coupled, for instance by one or more support arms 28. The inner rim 24 and the outer rim 26 can be circular and can be arranged concentrically. As used herein, “circular” includes both circle-shaped and elliptical perimeters and variations thereof, such as multi-sided polygons having a generally circular or elliptical shape. As used herein, “encircle” has its standard meaning. For example, the inner rim 24 is encircled by the outer rim 26 where the inner rim 24 does not extend beyond a three dimensional, e.g., cylindrical, projection of the outer rim 26. Thus, the outer rim 26 can encircle the inner rim 24 even where the two anterior rims 24, 26 are not coplanar. In some examples, the rims 24, 26 are coplanar, while in other examples the two anterior rims 24, 26 are not coplanar.

The islet storage feature 18 can take the form or one or more pouches, two or more pouches, or even three or more pouches. As shown in FIGS. 1-4, the islet storage feature 18 can include two pouches 30. The pouches 30 can extend around 300 degrees or more of the perimeter of the iris, as measured at the anterior portion 12 of the implant 10. The islet storage feature can have an interior volume of at least 0.75 cm³, at least 1.0 cm³, at least 1.25 cm³, or at least 1.5 cm³.

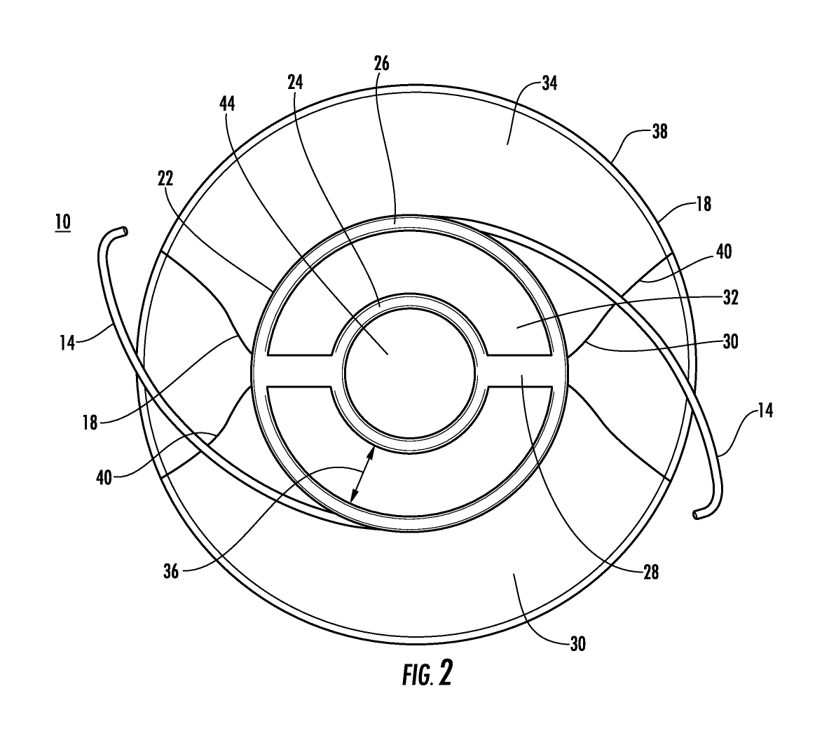

As shown in FIG. 2, an inner portion 32 of the islet storage feature 18 can be coupled to the inner rim 24 and an outer portion 34 of the islet storage feature 18 can be coupled to the outer rim 26. The interior 20 of the islet storage feature 18 can be accessible through at least one space 36 between the inner rim 24 and the outer rim 26. The islet storage feature 18 can be closed, i.e., sealed, at the posterior portion 16. As used herein, “coupled” has its normal meaning and can include attached.

The islet storage feature 18 can include a permeable material, such as a film. The islet storage feature can include a biocompatible, flexible material, including, but not limited to, polymethyl methacrylate (PMMA), acrylic polymers, styrene-isobutene-styrene (SIBS) co-polymers and polydimethylsiloxane (PDMS), mixtures thereof and co-polymers thereof. The inner and outer portions 32 and 34 can include films coupled to a shaping rim 38 at one end and at the inner rim 24 and outer rim 26, respectively, at the opposite end. The shaping rim 38 can be a circular hoop designed to minimize any interference of the islet storage features 18 with vision. The diameter of the shaping rim 38 can be 10-75 mm or 20-50 mm.

The inner and outer portion 32, 34 can be formed from the same film, i.e., can be looped around the shaping rim 38 and start and end at the anterior portion 12. The lateral edges 40 of the islet storage features 18 can be formed by joining the inner and outer portions 32, 34 together to form a pouch. For example, the lateral edges 40 can be joined using an adhesive or binding, e.g., a clip or thread, or cohesively bonded, e.g., thermal bonding and ultrasonic bonding. Alternately, the lateral edges 40 can be formed by folding a single piece of film that forms the inner and outer portions 32, 34. The material forming the inner and outer portions 32, 34 can be liquid permeable, oxygen permeable, protein permeable and cytokine permeable.

Where the inner and outer portions 32, 34 include pores, the pores should be designed to retain islets within the islet storage feature 18, while also being large enough to allow proteins and cytokines to pass through the inner and outer portions 32, 34. In general, the inner and outer portions 32, 34 can include pores that range from 50 nm to 10 μm, or 100 nm to 5 μm, or 250 nm to 2 μm. As will be understood, the islets maintained within the storage feature 18 can be present as free floating single cells, cells engrafted on a scaffold, or as agglomerations of cells.

The intraocular implant 10 can also include an optical element 44, such as an intraocular lens. The optical element 44 can be coupled to the anterior portion 12, for example, the core 22 of the anterior portion 12, or more particularly, to the inner rim 24. In order to avoid interference with the patient's visions, the islet storage feature 18 can be arranged so that the islet storage feature 18 does not extend into an optical cavity 42 of the intraocular implant 10. The optical cavity 42 can be defined as a three-dimensional projection from the perimeter of optical element 44. For example, the optical cavity 42 can be a cylinder.

The invention is also drawn to an intraocular implant kit that includes the intraocular implant 10 described herein disposed within a cannula. As used herein, cannula is defined in its broadest sense and includes catheters. The intraocular implant kit can include an optical element 44, such as an intraocular lens, separate from or incorporated into the intraocular implant 10. The intraocular implant 10 can be formed of resilient materials that can be collapsed within the cannula, but that return to the desired shape and orientation once deployed from the cannula. Exemplary materials that can be used as components of the intraocular implant include, but are not limited to, elastomers (soft polyacrylics, polysiloxanes, poly(hydroxyethylmethacrylates), etc.), nitinol (Nickel Titanium) alloys, polyfluoropolyether (PFPE), or other super-elastic or shape-memory alloys. The intraocular kit can also include a plurality of islets to be deposited in the islet storage feature(s) 18 after the intraocular implant has be deployed from the cannula.

A method of treating a disease state is also described. The disease state being treated can be any disease treated by intraocular implant and/or islet transplant including, but is not limited to, one or more of the following: diabetes, diabetic retinopathy, macular degeneration, macular edema, and other blinding eye diseases.

The method can include implanting an intraocular implant 10 into the eye of a patient. The intraocular implant 10 can include an anterior portion 12, a posterior portion 16 and an islet storage feature 18 coupled to the anterior portion 12 and the posterior portion 16. An interior portion 20 of the islet storage feature 18 can be accessible from the anterior portion 12. The method can include depositing a plurality of islets into the islet storage feature 18 of the intraocular implant 10 and/or coupling an optical element 44 to the anterior portion 12 of the intraocular implant 10. For example, the optical element 44 can be snapped into a portion, e.g., the inner rim 24, of the anterior portion 12 designed to receive the optical element 44. Alternately, the optical element 44 can be attached to the anterior portion 12 via polymeric fusion. The depositing and coupling steps can occur before or after the intraocular implant 10 is implanted in an eye of the patient. The intraocular implant 10 can be implanted using a cannula.

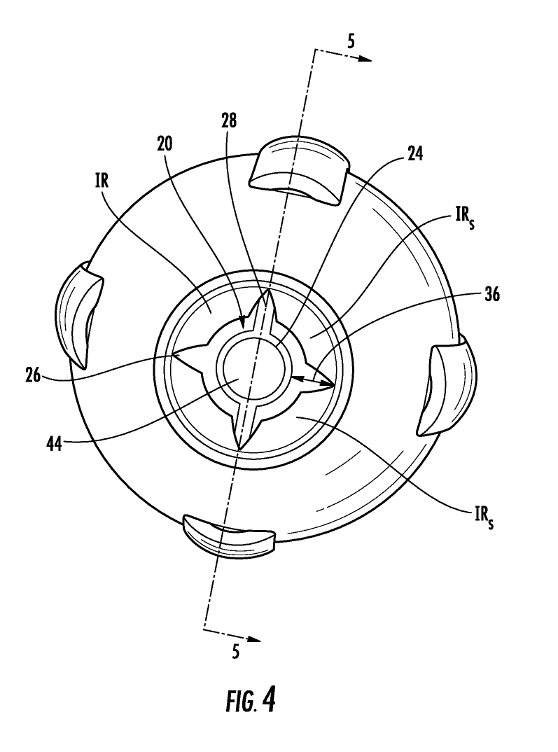

The method can also include cutting an iris (IR) of the eye to form at least two iris segments 48. As shown in FIGS. 4-5, a portion of the at least two iris segments (IRA four in this case, can be located or placed in the interior of the islet storage feature(s) 18. The anterior portion 12 can include an inner rim 24 coupled to an outer rim 26 that encircles the inner rim and the at least two iris segments can extend through the space(s) 36 between the inner and outer rim, 24, 26.

The outer edge of the iris, known as the root, is attached to the sclera and the anterior ciliary body. The iris and ciliary body together are known as the anterior uvea. The iris is divided into two major regions:

The pupillary zone is the inner region whose edge forms the boundary of the pupil.

The ciliary zone is the rest of the iris that extends to its origin at the ciliary body.

The collarette is the region of the iris separating the pupillary portion from the ciliary portion. It is typically defined as the region where the sphincter muscle and dilator muscle overlap.

With this as background, the cutting step can include cutting the iris (IR) without cutting the root of the iris (IR). The cutting step can include cutting completely through the papillary zone or cutting through some, but not all, of the papillary zone. Generally, the direction of the cuts will be radial, i.e., projecting outward from the center of the pupil. Placing the iris in close proximity to the islets provides the islets with a blood supply and oxygen source, which provides for extended life of the islets disposed within the islet storing intraocular implant. In general, the islets will engraft to the iris after implantation. This is one of the many reasons to avoid cutting the root of the iris.

In a particular method of treating a disease state, the disease state can be diabetic retinopathy. The method can include phacoemulsion of the crystalline lens, removal of the posterior capsule and a total vitrectomy. The intraocular implant can then be inserted through a corneal incision using a cannula. The haptics can be deployed and located in the sulcus (S) of the eye. An intraocular lens can be snapped into the inner rim of the implant and islets can be injected into the islet storage feature. The iris can then be segmented into two halves or four quadrants without cutting the root. Each of the iris sections can be pushed through spaces between the inner and outer rims and into the interior of the islet storage feature. A bandage contact can then be placed on the cornea to protect the incision, which can also be sutured depending on the patient, e.g., likelihood of eye rubbing, and the specific circumstances of the procedure, e.g., incisions>3 mm.

The function and advantage of these and other embodiments of the present invention will be more fully understood from the examples described below. The following examples are intended to facilitate an understanding of the invention and to illustrate the benefits of the present invention, but are not intended to limit the scope of the invention.

### EXAMPLES

A rodent model has been used to demonstrate that the anterior chamber of the eye is an optimal islet implantation site allowing pancreatic islets to engraft and function indefinitely and to be monitored non-invasively (Speier et al., 2008A, B). This Example evaluates the eye as a safe site for therapeutic implantation in a relevant preclinical animal model. For this purpose baboons were rendered diabetic with streptozotocin (90 mg/kg), using the techniques disclosed in Berman et al., 2007, 2009. Allogeneic donor baboon islets, isolated and maintained as previously described (e.g., Kenyon et al., 1999, Berman et al., 2007), were transplanted into the anterior chamber of the right eye of the recipient baboon by performing paracentesis/corneal self-sealing incisions alternately at superior temporal and nasal quadrants. After draining the aqueous humor from the anterior chamber, ˜18,000 islet equivalents (˜2,100 islet equivalents/kg) were injected using an 18G cannula, ensuring that islets were distributed through the surface area of the iris. An air bubble was left in the anterior chamber to prevent collapsing of the cornea. After closing the wound with a 10-0 nylon suture, the animal was kept flat face up for 1 hour and was treated with topical steroids and topical antibiotics for 4 days post-implant. The monkey immunosuppression treatment was with anti-CD154 (5C8) at 20 mg/kg IV on post-operative days (PODs) −1, 0, 3, 10, 18, 28 and every 28 days thereafter.

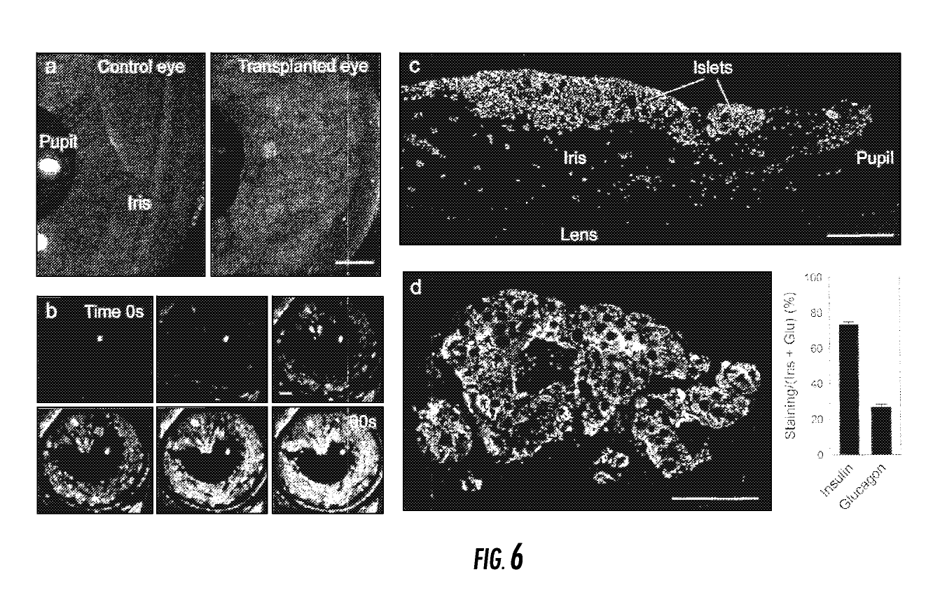

Islets engrafted on the iris, covering ˜80% of the iris surface (FIG. 6). Over the 357 days the experiment lasted there was little loss of islet mass (<15%). Angiographic studies showed that islet grafts were strongly vascularized as early as post-operative day (POD) 24 (FIG. 6b). FIG. 6c shows confocal images of a section of the anterior segment of the eye showing engraftment of islets on the iris. Insulin-labeled beta cells (red) and glucagon-labeled alpha cells (cyan) formed a distinct layer fully fused with the iris tissue. The cytoarchitecture of transplanted islets was preserved, and the cellular composition was similar to that of the islets prior to transplantation (FIG. 6d).

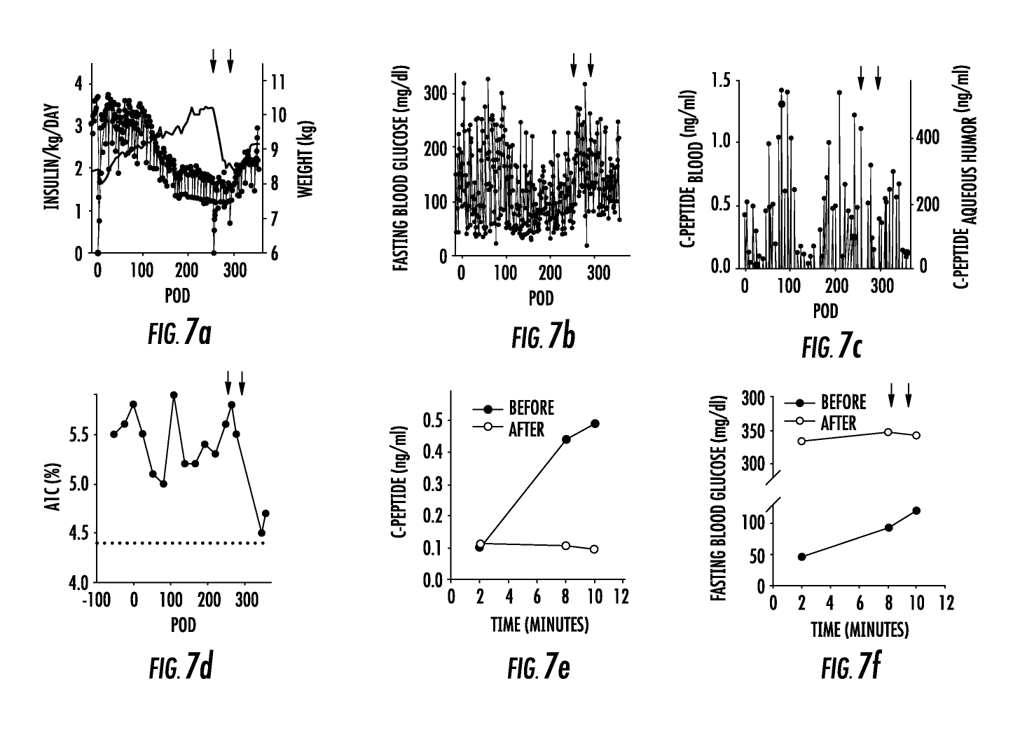

Graft function was monitored using the following clinical parameters: pre- and post-transplant exogenous insulin requirements, HbA1c (glycated hemoglobin, a measure of the average plasma glucose concentration over prolonged periods of time), fasting blood glucose levels, the ratio of C-peptide to fasting blood glucose levels, and C-peptide secretion after intravenous glucose and glucagon challenges. Exogenous insulin requirements started decreasing at 3 months after transplantation and stayed lower (−60% of pretransplant requirements) for the duration of the experiment (FIG. 7a). In parallel with the reduction of insulin requirements, fasting glycemia became less erratic, indicating improved and more stable metabolic control (FIG. 7b). C-peptide levels, a measure of endogenous insulin production, exceeding 1 ng/ml could be measured in the circulation at 3 months after transplantation. Very high levels of C-peptide (>500 ng/ml) were detected in the aqueous humor of the transplanted eye, and these values co-varied with blood C-peptide values (FIG. 7c). Increases in C-peptide levels could be induced in response to intravenous glucose challenges, and the magnitude of the response increased progressively with time after transplantation. The glucose excursions in these tests further indicated that glycemia was actively controlled. HbA1c decreased gradually after transplantation to reach a nadir at 3 months. Intraocular islet grafts thus manifestly contributed to glucose homeostasis and improved glycemic control. As shown in FIG. 7d, glycated hemoglobin (A1C) levels decreased after islet transplantation and, after the second islet transplantation, almost reached levels measured in the same baboon before diabetes was induced (dotted line).

To eliminate a putative contribution of residual insulin secretion by endogenous islets surviving the streptozotocin treatment, we pancreatectomized the baboon on POD 256. While transient changes in glycemia and weight loss were associated with the procedure, C-peptide was still detectable in the circulation (>0.5 ng/ml; FIG. 7c) and its secretion could be stimulated with glucose (FIG. 7e), confirming that intraocular islet grafts were fully functional and had indeed contributed most of the detectable blood C-peptide. To further improve glycemic control, we performed a second allogeneic islet transplantation into the same eye on POD 292. Of the ˜18,000 islet equivalents (2100 islet equivalents/kg) injected into the eye, only few could be seen engrafting on the first layer of previously engrafted islets. Nevertheless, compared to post-pancreatectomy values, C-peptide levels increased after the second islet transplantation (FIG. 7c) Importantly, HbA1c levels continued to decrease, attaining lowest values at POD 347.

The experiment was terminated at the first signs of islet rejection (i.e. a sharp increase in insulin requirements and fasting blood glucose). At the day of necropsy, in this case POD 357, glucagon challenges were performed before and after removal of the transplanted eye. In this definitive test for islet graft function, C-peptide secretion could be elicited and detected before but not after removing the eye with islet grafts (FIG. 7e) and the baboons returned to hyperglycemia immediately after removal of the eye (FIG. 7f).

Over the course of the experiment, eight complete examinations of the transplanted, right eye and of the not transplanted, left eye were conducted using slit lamp exams and intraocular pressure measurements by tonopen. There was no difference in the intraocular pressure between the eyes at any time point (7.6±1.3 mm/Hg versus 9.7±1; average±SEM), which is consistent with the absence of glaucoma and the observation that the angle (i.e., the trabecular meshwork) of the anterior segment was always clear and open.

The cornea of the transplanted eye was always clear and the conjunctiva and aqueous humor were quiet, that is, there were no signs of intraocular inflammation or endophthalmitis. At POD 7, a small portion of the iris started adhering to the lens (posterior synechia) due to iatrogenic trauma that occurred when the islet injection cannula inadvertently hit the crystalline lens and potentially, the iris, but the synechia stabilized thereafter. It was only after the second islet transplantation, at POD 309, that we observed an anterior capsule cataract. There was no pathological neovascularization in either the anterior or posterior portions of the eye, and the optic nerve and the retina remained normal throughout the study.

Given the difficulties with current implantation sites in connection with clinical islet transplantation as a mean to treat patients with type 1 diabetes, the immune-privileged anterior chamber of the eye was evaluated as a therapeutic transplantation site. The data demonstrates that pancreatic islets engraft and function in the eye of a diabetic baboon, contributing to glycemic control. The absence of ophthalmological complications demonstrates the anterior chamber of the eye is a promising location for the translation to beta-cell replacement in humans, in particular those with already compromised vision (i.e., diabetic retinopathy).

Based on these results, it can be determined that the islets survived and produced insulin because they were fed by the iris blood circulation and bathed by aqueous humor. The intraocular implants and implantation methods described herein will provide viable islets because removal of the crystalline lens and the vitreous will not affect islet survival but will permit a larger number of cells to be implanted and thus increase total insulin production.

While there has been shown and described what are at present considered the preferred embodiments of the invention, it will be obvious to those skilled in the art that various changes and modifications can be prepared therein without departing from the scope of the inventions defined by the appended claims.

1. An intraocular implant, comprising:
an anterior portion,
at least one haptic element designed to localize said intraocular implant within an eye;
a posterior portion; and
an islet storage feature coupled to said anterior portion and said posterior portion

2. The intraocular implant according to claim 1, wherein an interior of said islet storage feature is accessible from said anterior portion.

3. The intraocular implant of claim 1, wherein said at least one haptic element comprises at least two resilient arms extending from a core of said anterior portion.

4. The intraocular implant of claim 1, wherein said anterior portion comprises an inner rim coupled to an outer rim encircling said inner rim, wherein an interior of said islet storage feature is accessible through at least one space between said inner rim and said outer rim.

5. The intraocular implant of claim 4, wherein said inner rim and said outer rim are circular and arranged concentrically.

6. The intraocular implant of claim 4, wherein an inner portion of said islet storage feature is coupled to said inner rim and an outer portion of said islet storage feature is coupled to said outer rim.

7. The intraocular implant of claim 1, wherein said islet storage feature is closed at said posterior portion.

8. The intraocular implant of claim 1, wherein said islet storage feature comprises a permeable material.

9. The intraocular implant of claim 1, wherein said islet storage feature does not extend into an optical cavity of said intraocular implant.

10. The intraocular implant according to claim 1, further comprising an optical element.

11. An intraocular implant kit, comprising:
an intraocular implant according to claim 1 disposed within a cannula.

12. The intraocular implant kit according to claim 11, further comprising:
an optical element.

13. The intraocular implant kit according to claim 12, wherein said optical element is an intraocular lens.

14. The intraocular implant kit according to claim 11, further comprising a plurality of islets.

15. A method of treating a disease state, comprising:
implanting an intraocular implant into an eye of a patient, wherein said intraocular implant comprises an anterior portion, a posterior portion and an islet storage feature coupled to said anterior portion and said posterior portion; and depositing a plurality of islets into an interior of said islet storage feature.

16. The method of treating a disease state according to claim 15, wherein said implanting step comprises, implanting said intraocular implant using a cannula.

17. The method of treating a disease state according to claim 15, further comprising, coupling an optical element to said anterior portion.

18. The method of treating a disease state according to claim 15, further comprising, cutting an iris of said eye to form at least two iris segments, and locating a portion of said at least two iris segments in said interior of said islet storage feature.

19. The method of treating a disease state according to claim 18, wherein said cutting step comprises cutting said iris without cutting to a root of said iris.

20. The method of treating a disease state according to claim 19, wherein said anterior portion comprises an inner rim coupled to an outer rim encircling said inner rim element, and said interior is accessible through at least one space between said inner rim and said outer rim and said at least two iris segments extend through said at least one space.

21. The method according to claim 15, wherein said disease state is selected from the group consisting of diabetes, diabetic retinopathy.
